(19) United States
(12) Patent Application Publication
(10) Pub. No.: US 2009/0123436 A1
(43) Pub. Date: May 14, 2009

(54) CRYOPRESERVATIVE COMPOSITIONS AND METHODS

(75) Inventor: Gary W. Opperman, St. Louis Park, MN (US)

Correspondence Address: Pauly, Devries Smith & Deffner, L.L.C., Plaza VII, 45 South Seventh Street, Suite 3000, Minneapolis, MN, 55402-1630, US

(73) Assignee: SurModics, Inc., Eden Prairie, MN (US)

(21) Appl. No.: 12/266,955

(22) Filed: Nov. 7, 2008

Related U.S. Application Data
(60) Provisional application No. 60/986,335, filed on Nov. 8, 2007.

(51) Int. Cl. A61K 35/12; C12N 5/06

(52) U.S. Cl. 424/93.7; 435/325; 435/374

(57) ABSTRACT

The invention relates to compositions for the cryogenic storage of biological materials and related methods. In an embodiment, the invention includes a cryopreservative composition including a chaotropic agent and a kosmotropic agent. In an embodiment, the invention includes a cryopreservative composition including urea and trimethylamine-N-oxide. In an embodiment, the invention includes a method of cryopreserving cells including contacting cells with a cryopreservative composition, the cryopreservative composition comprising a chaotropic agent and a kosmotropic agent. In an embodiment, the invention includes a method of transplanting cells into a subject, the method including administering a composition to the subject, the composition comprising an effective amount of a chaotropic agent, an effective amount of a kosmotropic agent, and cells. Other embodiments are also included herein.

This application claims the benefit of U.S. Provisional Application No. 60/986,335, filed Nov. 8, 2007, the contents of which are herein incorporated by reference.

### FIELD OF THE INVENTION

The invention relates to compositions for the cryogenic storage of biological materials and related methods.

### BACKGROUND OF THE INVENTION

Cryopreservation is a process where biological samples such as cells or whole tissues are preserved by cooling to low sub-zero temperatures. At such low temperatures, any biological activity, including the biochemical reactions that would normally lead to cell death, is effectively stopped. Cryopreservation has many different research and clinical applications. By way of example, there is a frequent research need to store cell or tissue samples for a period of time in a manner so as to preserve their potential for resuming biological activity, such as in the cases of cell culture samples and hybridomas. In addition, there is a frequent clinical need to preserve and to store cells while preserving their potential biological activity, such as in the case of autologous bone marrow transplants, cord blood storage, and the storage of human gametes.

Cooling cells and/or tissues to sub-zero temperatures can potentially cause significant damage to the cells and/or tissues such that biological activity cannot be resumed after elevating temperature back to a normal level. For example, ice crystals can form which physically disrupt cell membranes, leading to cell death.

One technique for maintaining the biological activity of cell samples in the context of cryopreservation is the use of dimethyl sulfoxide (DMSO) as a cryoprotective agent that functions to increase the number of cells that survive both the cooling process and the subsequent heating process. DMSO is generally used at a final concentration of 5% to 15% (v/v) (0.74 to 2.5 molal). DMSO is believed to work at least in part by disrupting the process of ice crystal formation, thereby reducing the physical disruption of cell membranes. However, DMSO can cause various side effects. For example, patients who receive autologous cell transplants that have been preserved in DMSO can experience various side effects including headaches, nausea and skin rash. (Davis, J M et. al., Blood, 75(3), 1990, pp 781-786) In addition, some cell lines are adversely affected by prolonged contact with DMSO.

Another technique for maintaining the biological activity of cell samples in the context of cryopreservation is the use of glycerol as a cryoprotective agent. Glycerol is generally used at a final concentration of between 5 and 20% (v/v). Although glycerol is generally less toxic to cells than DMSO, glycerol can cause osmotic problems, especially after thawing.

Therefore, a need exists for compositions useful for the cryogenic storage of biological materials and related methods.

### SUMMARY OF THE INVENTION

The invention relates to compositions for the cryogenic storage of biological materials and related methods. In an embodiment, the invention includes a cryopreservative composition including a chaotropic agent and a kosmotropic agent.

In an embodiment, the invention includes a cryopreservative composition including urea and trimethylamine-N-oxide.

In an embodiment, the invention includes a method of cryopreserving cells including contacting cells with a cryopreservative composition, the cryopreservative composition comprising a chaotropic agent and a kosmotropic agent.

In an embodiment, the invention includes a method of transplanting cells into a subject, the method including administering a composition to the subject, the composition comprising an effective amount of a chaotropic agent, an effective amount of a kosmotropic agent, and cells.

The above summary of the present invention is not intended to describe each discussed embodiment of the present invention. This is the purpose of the figures and the detailed description that follows.

### BRIEF DESCRIPTION OF THE DRAWINGS

The invention may be more completely understood in connection with the following drawings, in which:

FIG. 1 is a graph of cell viability after cryopreservation as evaluated with a cooling rate of 10 degrees Celsius per minute.

FIG. 2 is a graph of cell viability after cryopreservation as evaluated with a cooling rate of 20 degrees Celsius per minute.

FIG. 3 is a graph of cell yield after cryopreservation as evaluated with a cooling rate of 10 degrees Celsius per minute.

FIG. 4 is a graph of cell yield after cryopreservation as evaluated with a cooling rate of 20 degrees Celsius per minute.

While the invention is susceptible to various modifications and alternative forms, specifics thereof have been shown by way of example and drawings and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Embodiments of the invention can include cryopreservative compositions that serve to provide effective cryopreservation while exhibiting limited toxicity to cells and tissues. In an embodiment, the invention can include a cryopreservative composition including a chaotropic agent and a kosmotropic agent.

The term “chaotrope” or “chaotropic agent” as used herein shall refer to any substance that increases the transfer of apolar groups to water because of its ability to decrease the ordered structure of water and to increase its lipophilicity. Chaotropes tend to destabilize proteins and membranes. Chaotropes can also be referred to as “denaturing osmolytes”.

Exemplary chaotropic agents used with embodiments of the invention can include urea and guanidinium chloride, amongst others. In some embodiments, the chaotropic agent is urea.

The term “kosmotrope” or “kosmotropic agent” as used herein shall refer to any substance that decreases the transfer of apolar groups to water because of its ability to increase the ordered structure of water and to decrease its lipophilicity. Kosmotropes tend to stabilize proteins and membranes. Kosmotropes can also be referred to as “protecting osmolytes”.

Exemplary kosmotropic agents used with embodiments of the invention can include trimethylamine N-oxide (TMAO), proline, ectoine, α,α-trehalose, glycine betaine, 3-dimethylsulfoniopropionate, sarcosine, sucrose, and sorbitol.

Beyond chaotropes and kosmotropes, cryopreservative compositions of the invention can also include various additives. By way of example, exemplary additives can include water soluble polymers, antibiotics, proteins, salts, sugars, dyes, and the like.

The effectiveness of cryopreservation can be characterized, in part, by the relative number of cells that survive the cryopreservation process. Cellular survival can, in turn, be characterized by various measures including cell yield and viability. As used herein, the term “cell yield” shall refer to the number of cells that are live after the cryopreservation process divided by the number of cells that were live before the cryopreservation process. Cell yield can be expressed as a fraction (0.0-1.0) or a percent. It will be appreciated that the number of live cells can be ascertained in various ways known to those of skill in the art including cell staining. As used herein, the term “viability” shall refer to the number of cells that are live after the cryopreservation process divided by the total number of cells that can be counted after the cryopreservation process. Viability can also be expressed as a fraction (0.0-1.0) or a percent.

As shown in the examples below, compositions and methods of the invention can be used to achieve relatively high levels of both cell yield and viability. In some embodiments, compositions and methods of the invention can be used to achieve cell yield on a fractional basis of equal to or greater than about 0.2. In some embodiments, compositions and methods of the invention can be used to achieve cell yield on a fractional basis of equal to or greater than about 0.4. In some embodiments, compositions and methods of the invention can be used to achieve cell yield on a fractional basis of equal to or greater than about 0.6. In some embodiments, compositions and methods of the invention can be used to achieve cell yield on a fractional basis of equal to or greater than about 0.8.

In some embodiments, compositions and methods of the invention can be used to achieve viability on a fractional basis of equal to or greater than about 0.2. In some embodiments, compositions and methods of the invention can be used to achieve viability on a fractional basis of equal to or greater than about 0.4. In some embodiments, compositions and methods of the invention can be used to achieve viability on a fractional basis of equal to or greater than about 0.6. In some embodiments, compositions and methods of the invention can be used to achieve viability on a fractional basis of equal to or greater than about 0.8.

It is believed that the relative proportions of the kosmotropic agent to the chaotropic agent can influence cell survival. One way of describing the proportion of kosmotropic agents to chaotropic agents is by mole fraction. As used herein, mole fraction shall refer to the fraction of moles of a kosmotropic or chaotropic agent in comparison to the total moles of both the chaotropic and kosmotropic agents in the composition. As such mole fraction shall be determined according to the following formulas:

MF_{Chaotrope}=[A]/([A]+[B])

MF_{Kosmotrope}=[B]/([A]+[B])

wherein [A]=the number of moles of the chaotrope in the composition and [B]=the number of moles of the kosmotrope in the composition.

Embodiments of the invention can include cryopreservative compositions with a kosmotrope mole fraction of greater than zero and less than one. In some embodiments, the kosmotrope mole fraction can be between about 0.1 and 0.7. In some embodiments, the kosmotrope mole fraction can be between about 0.1 and 0.5. In an embodiment, the kosmotrope mole fraction can be between 0.1 and 0.35.

As shown in the examples below, the total amount of kosmotropic agents and chaotropic agents in the cryopreservative composition can affect both viability and cell yield. Because of the substantial changes in temperature associated with cryopreservation techniques and the resulting changes in fluid volumes, one convenient way to assess concentrations of composition components is with units of molality. Molality is defined as the number of moles of solute per kilogram of solvent. Embodiments of cryoprotective compositions can include a total cryoprotective agent concentration (kosmotropic agents and chaotropic agents combined) of between about 0.01 Molal and 1.5 Molal. In some embodiments, the cryoprotective composition can include a total cryoprotective agent concentration of between about 0.1 Molal and 1.0 Molal. In some embodiments, the cryoprotective composition can include a total cryoprotective agent concentration of between about 0.1 Molal and 0.5 Molal.

Embodiments of the invention can include methods of cryogenically preserving a sample. Such methods can include a step of contacting cells with a cryopreservative composition, the cryopreservative composition comprising a chaotropic agent and a kosmotropic agent. In some embodiments, cryopreservation methods of the invention can include initial cell preparation, addition of a cryopreservative composition to the cells, followed by cooling of the cells at a defined cooling rate. Embodiments can also include storage of the cells at a defined temperature followed by warming of the cells at a defined warming rate.

The rate of cooling is a significant parameter affecting cell survival in cryopreservation methods. Cooling rates that are too high (too rapid) can result in a reduced rate of cell survival. It is believed that high cooling rates can be harmful to cell survival because of the formation of ice inside of cells. Cooling rates that are too low (too slow) can also result in a reduced rate of cell survival. It is believed that low cooling rates can be harmful to cell survival through excessive cellular dehydration. The optimum rate of cooling is cell type dependent. It is believed that water transport properties of the cell membrane are the primary factor in the cell dependence of the optimum cooling rate.

As shown herein, cryopreservative compositions of embodiments of the invention can be successfully used with relatively high cooling rates to achieve high cell survival rates. In an embodiment, the cooling rate using cryopreservative compositions as described herein can be equal to or greater than about 5 degrees Celsius per minute. In an embodiment, the cooling rate using cryopreservative compositions as described herein can be equal to or greater than about 10 degrees Celsius per minute. In an embodiment, the cooling rate using cryopreservative compositions as described herein can be equal to or greater than about 20 degrees Celsius per minute. In some embodiments, the cooling rate using cryopreservative compositions as described herein can be between about 5 degrees Celsius per minute and about 40 degrees Celsius per minute. In some embodiments, the cooling rate using cryopreservative compositions as described herein can be between about 10 degrees Celsius per minute and about 30 degrees Celsius per minute.

Various types of equipment can be used to achieve specific cooling rates. By way of example, one exemplary freezing system is the BIOCOOL IV controlled rate freezer, available commercially from FTS Systems, Inc., Stone Ridge, N.Y. Another exemplary freezing system is the Model 2100 Controlled Rate Freezing System, available commercially from Custom BioGenic Systems, Shelby Township, Mich. Many other types of freezing systems can also be used.

Storage of the cells can include various defined temperatures for various lengths of time. In some embodiments, cells are stored for a period of time greater than about two hours. In some embodiments, cells are stored for a period of time greater than two days. In some embodiments, cells are stored for a period of time greater than about one month. In some embodiments, cells are stored for a period of time greater than about six months.

Various storage temperatures can be used depending on the type of cells and other factors. In some embodiments, the storage temperature is less than about −50 degrees Celsius. In some embodiments, the storage temperature is less than about −100 degrees Celsius. In some embodiments, the storage temperature is less than about −150 degrees Celsius.

The rate of warming is believed to be a significant parameter affecting cell survival in cryopreservation methods. Warming rates that are too high (too rapid) can result in a reduced rate of cell survival. Similarly warming rates that are too low (too slow) can also result in a reduced rate of cell survival. In some embodiments, the warming rate using cryopreservative compositions as described herein can be equal to or greater than about 10 degrees Celsius per minute. In an embodiment, the warming rate using cryopreservative compositions as described herein can be equal to or greater than about 50 degrees Celsius per minute. In an embodiment, the warming rate using cryopreservative compositions as described herein can be equal to or greater than about 100 degrees Celsius per minute.

Cryoprotective compositions described herein can include those exhibiting minimal toxicity. This can be desirable as preserved cells may be administered to a subject and even repeated washing steps of the cell sample may fail to remove all traces of cryoprotective agents. Embodiments of the invention can specifically include methods of transplanting cells into a subject. By way of example, the method can include administering a composition to the subject, the composition comprising an effective amount of a chaotropic agent, an effective amount of a kosmotropic agent, and cells.

The present invention may be better understood with reference to the following examples. These examples are intended to be representative of specific embodiments of the invention, and are not intended as limiting the scope of the invention.

### EXAMPLES

### Example 1

### Toxicity of Cryopreservative Compositions

A test cryopreservative composition was prepared by adding urea and TMAO to an HTS (HYPOTHERMOSOL FRS) solution (BioLife Solutions, Seattle, Wash.) to result in concentrations of 0.5 Molal urea and 0.5 Molal TMAO. HYPERTHERMOSOL has been reported to include Na⁺ (100 mM), K⁺ (42.5 mM), Ca²⁺ (0.05 mM), Mg²⁺ (5.0 mM), Cl⁻ (17.1 mM), H₂PO₄ (10 mM), HCO₃ (5.0 mM), HEPES (25.0 mM), lactobionate (100.00 mM), sucrose (20.0 mM), mannitol (20.0 mM), glucose (5.0 mM), Dextran-40 (6.0%), adenosine (2.0 mM), and glutathione (3.0 mM). See Baust et al., Modulation of the cryopreservation cap: elevated survival with reduced dimethyl sulfoxide concentration, Cryobiology, (2002) 45:97-108.

A comparative cryopreservative composition was prepared by adding DMSO to an HTS solution to result in a concentration of 5% DMSO (or approximately 0.7 Molal).

Samples of Jurkat cells (ATCC # TIB-132) obtained from the American Type Culture Collection (ATCC) were counted and then put in aliquots of approximately 1×10⁶ cells in microcentrifuge tubes and spun down in a centrifuge. The supernatants were then discarded. Next, the cells were resuspended in either the test cryopreservative composition or the comparative cryopreservative composition (1 ml). The samples were then incubated at 37 degrees Celsius for a period of four hours. Next, the number of viable cells was assessed by staining with 0.02 g/L propidium iodide and 0.02 mM acridine orange in phosphate buffered saline and comparing the number of viable cells before and after the incubation period. The data show that the percentage of viable cells after four hours in the test cryopreservative composition was 78% compared to only 54% for the comparative cryopreservative composition. As such, this example shows that cryopreservative compositions of some embodiments of the invention are less toxic to certain cell types than DMSO compositions.

### Example 2

### Protective Effects of Cryopreservative Compositions

A series of test solutions were prepared by adding urea and TMAO to a HTS (HYPOTHERMOSOL FRS) solution (BioLife Solutions, Seattle, Wash.) in various concentrations as shown below in Table 1.

TABLE 1

| | | | Total Cryoprotective | |
|---|---|---|---|---|
| | | | Agent | |
| Test | Concentration | Concentration | Concentration- | |
| Solution | of TMAO | of Urea | CPA (TMAO + | TMAO |
| # | (Moles/Kg) | (Moles/Kg) | Urea) (Moles/Kg) | Fraction |
| 1 | 0.0 | 0.0 | 0.0 | 0.0 |
| 2 | 0.02 | 0.08 | 0.1 | 0.2 |
| 3 | 0.033 | 0.066 | 0.1 | 0.33 |
| 4 | 0.06 | 0.24 | 0.3 | 0.2 |
| 5 | 0.1 | 0.2 | 0.3 | 0.33 |
| 6 | 0.0 | 0.5 | 0.5 | 0.0 |
| 7 | 0.1 | 0.4 | 0.5 | 0.2 |
| 8 | 0.165 | 0.335 | 0.5 | 0.33 |
| 9 | 0.25 | 0.25 | 0.5 | 0.5 |
| 10 | 0.335 | 0.165 | 0.5 | 0.67 |
| 11 | 0.5 | 0.0 | 0.5 | 1.0 |
| 12 | 0.33 | 0.67 | 1.0 | 0.33 |
| 13 | 0.5 | 0.5 | 1.0 | 0.5 |
| 14 | 0.5 | 1.0 | 1.5 | 0.33 |
| 15 | 1.0 | 0.5 | 1.5 | 0.67 |

A comparative cryopreservative composition was prepared by adding DMSO to RPMI 1640 (Invitrogen, Carlsbad, Calif.) with 10% fetal calf serum to result in a concentration of 10% (v/v) DMSO (or approximately 1.56 Molal).

Samples of Jurkat cells (ATCC # TIB-132) obtained from the ATCC were counted and then put in aliquots of approximately 1×10⁶ cells in microcentrifuge tubes and spun down in a centrifuge. The supernatants were then discarded. Next, the cells were resuspended in 1 ml of one of the test cryopreservative compositions or the comparative cryopreservative composition.

Each sample of Jurkat cells was then cryopreserved. Specifically, each sample of cells was cooled down to approximately −100° C. degrees using a Planer Kryo 10 Series 3 controlled rate freezer. Samples were cooled using the following cooling program:
1) Samples were put into the freezer at 20° C. and cooled to 0° C. at −10 C/min.
2) After 15 minutes at 0° C., samples were cooled to −8° C. at −1° C./min.
3) The extracellular volume was seeded by rapidly cooling to −45° C. at −50° C./min and then warming from −45° C. to −12° C. at 15° C./min.
4) The samples were then cooled from −12° C. to −100° C. at the cooling rate prescribed by the experiment. In this example some of the samples were cooled at a final rate of −10° C. per minute and some of the samples were cooled at a final rate of −20° C. per minute. It should be noted that standard procedures with DMSO frequently call for a final cooling rate of −1° C. per minute and accordingly those cell samples used with the comparative cryopreservative composition were cooled at a final rate of −1° C. per minute.

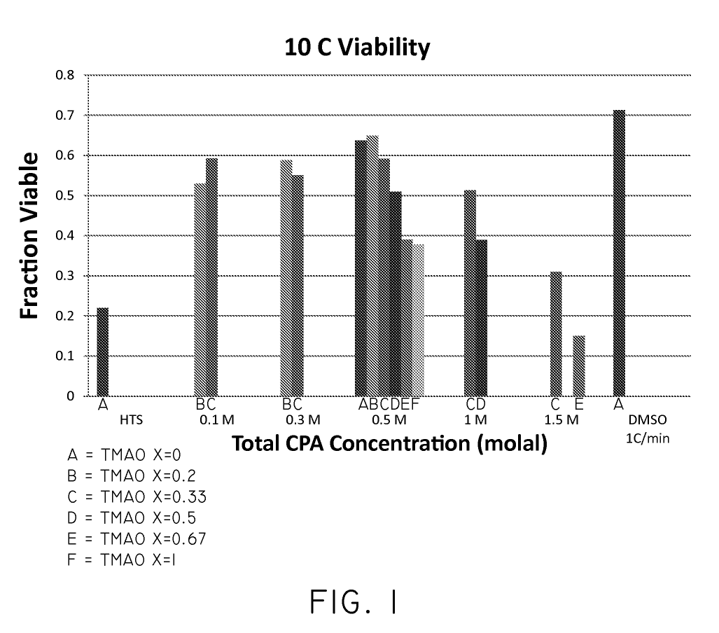

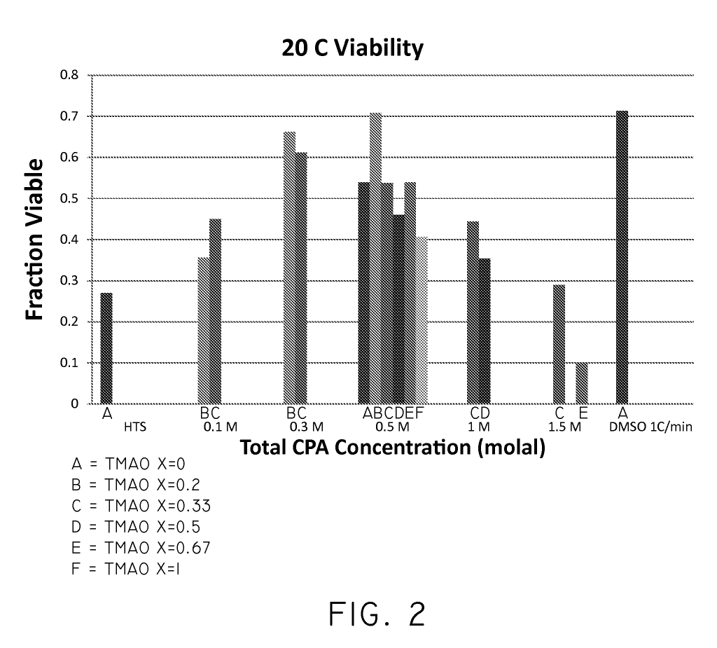

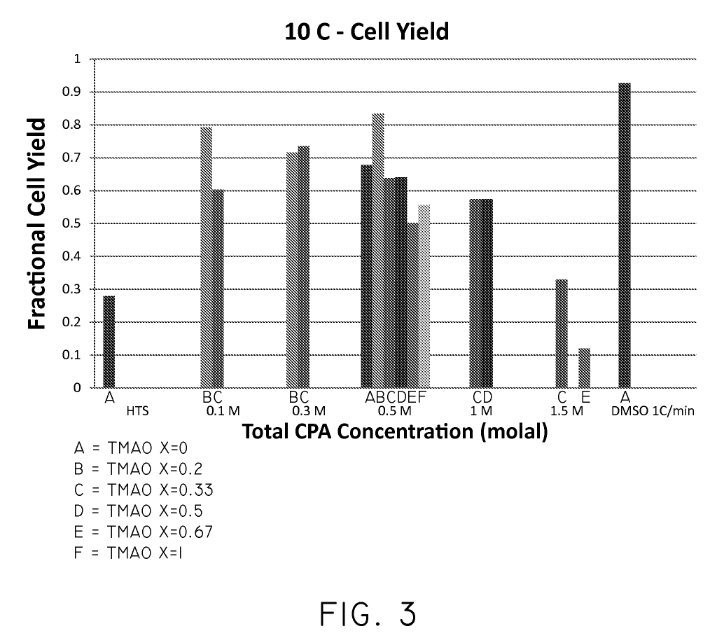

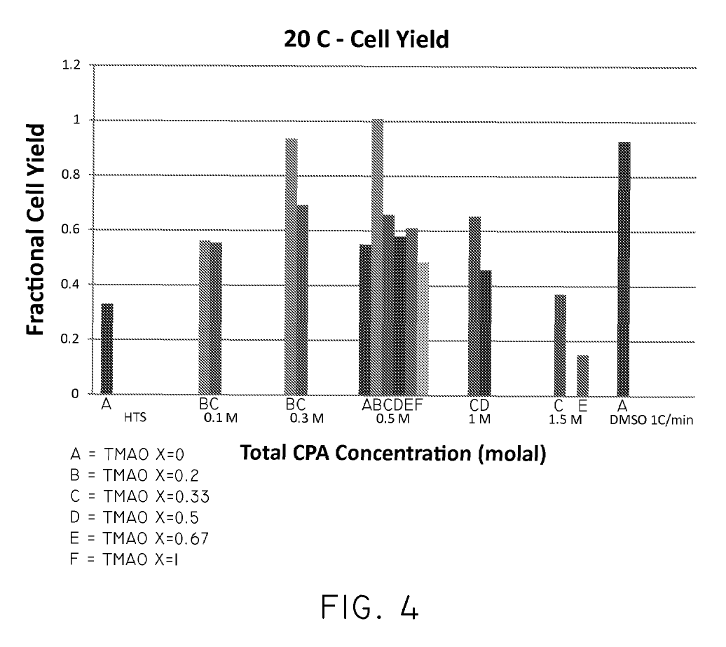

After storage in liquid nitrogen for approximately 2 weeks to 2 months, the cells were then warmed until thawed in a 37° C. water bath. The thawing time was from 2-3 minutes. The numbers of live and dead cells were then assessed by staining with 0.02 g/L propidium iodide and 0.02 mM acridine orange in phosphate buffered saline. Data for the samples, including both viability and yield are shown below in Tables 2-3. Data for the samples, including both viability and yield are shown in FIGS. 1-4.

TABLE 2

| | Viability | |
|---|---|---|
| Test Solution # | (Standard | Yield (Standard |
| (10 C./min) | Deviation) | Deviation) |
| 1 | 0.22 (0.045) | 0.28 (0.056) |
| 2 | 0.53 (0.033) | 0.792 (0.136) |
| 3 | 0.593 (0.026) | 0.603 (0.059) |
| 4 | 0.588 (0.034) | 0.716 (0.096) |
| 5 | 0.551 (0.044) | 0.735 (0.079) |
| 6 | 0.637 (0.034) | 0.678 (0.068) |
| 7 | 0.649 (0.046) | 0.834 (0.086) |
| 8 | 0.592 (0.021) | 0.638 (0.096) |
| 9 | 0.51 (0.054) | 0.64 (0.102) |
| 10 | 0.39 (0.013) | 0.5 (0.045) |
| 11 | 0.378 (0.078) | 0.556 (0.121) |
| 12 | 0.513 (0.061) | 0.574 (0.068) |
| 13 | 0.389 (0.028) | 0.5745 (0.121) |
| 14 | 0.31 (0.022) | 0.33 (0.047) |
| 15 | 0.15 (0.031) | 0.12 (0.023) |
| DMSO | 0.713 (0.100) | 0.926 (0.150) |

TABLE 3

| | Viability | |
|---|---|---|
| Test Solution # | (Standard | Yield (Standard |
| (20 C./min) | Deviation) | Deviation) |
| 1 | 0.27 (0.032) | 0.33 (0.038) |
| 2 | 0.356 (0.046) | 0.561 (0.026) |
| 3 | 0.45 (0.054) | 0.554 (0.087) |
| 4 | 0.662 (0.047) | 0.935 (0.078) |
| 5 | 0.612 (0.073) | 0.693 (0.114) |
| 6 | 0.539 (0.029) | 0.551 (0.049) |
| 7 | 0.708 (0.037) | 1.008 (0.193) |
| 8 | 0.537 (0.094) | 0.658 (0.052) |
| 9 | 0.46 (0.026) | 0.58 (0.087) |
| 10 | 0.54 (0.036) | 0.61 (0.095) |
| 11 | 0.407 (0.058) | 0.485 (0.046) |
| 12 | 0.444 (0.029) | 0.654 (0.038) |
| 13 | 0.354 (0.102) | 0.4585 (0.166) |
| 14 | 0.29 (0.036) | 0.37 (0.06) |
| 15 | 0.1 (0.011) | 0.15 (0.018) |
| DMSO | 0.713 (0.100) | 0.927 (0.150) |

The data show that cryoprotective compositions of the invention can be used to achieve relatively high viability and cell yield levels. The data also show that relatively low total concentrations of cryoprotective agents, such in the range of about 0.1 Molal and 0.5 Molal can be particularly effective.

### Example 3

### Protective Effect of Urea/TMAO Composition in Normal Saline

Urea and TMAO were dissolved in normal saline at concentrations of 0.4 Molal Urea and 0.1 Molal TMAO (similar to test solution #7 in example 2 above). This test solution was compared to normal saline for cryopreservation effectiveness. Freezing experiments were done as in example 2. Final cooling rates from −1° C./min to −40° C./min were investigated. Table 4 summarizes the results of this experiment.

TABLE 4

Fractional Cell Yield
(Standard Deviation)

| Cooling Rate | | Saline with |
|---|---|---|
| C./min | Normal Saline | TMAO/Urea |
| −1 | 0.05 (0.022) | 0.07 (0.024) |
| −10 | 0.03 (0.006) | 0.18 (0.045) |
| −20 | 0.03 (0.005) | 0.24 (0.036) |
| −30 | 0.04 (0.009) | 0.31 (0.018) |
| −40 | 0.04 (0.014) | 0.33 (0.034) |

The results show that cryoprotective compositions of the invention can vastly improve cell yield even in a very simple base solution. In other words, the beneficial effects of cryoprotective compositions in accordance with various embodiments of the invention are still evident even in the absence of solutions such as HYPOTHERMOSOL FRS as used in Example 2 above. This example also shows that cryoprotective compositions of the invention can be particularly beneficial at relatively high (fast) cooling rates.

### Example 4

### Protective Effect Using Betaine as the Kosmotrope

A solution was prepared by dissolving urea at a 0.4 Molal concentration and betaine at 0.1 Molal concentration in HTS. This solution was used in freezing experiments at cooling rates from 1 C/min to 40 C/min as described in example 2. Table 5 summarizes the results of this experiment.

TABLE 5

| | Urea/Betaine in HTS - |
|---|---|
| Cooling Rate | Fractional Cell Yield |
| ° C./min | (standard deviation) |
| −1 | 0.10 (0.014) |
| −10 | 0.72 (0.142) |
| −20 | 0.68 (0.094) |
| −30 | 0.66 (0.070) |
| −40 | 0.25 (0.099) |

The results show that using betaine to counteract the denaturing effects of urea works similarly to TMAO, thus betaine can be effectively applied as a kosmotrope in various embodiments herein.

It should be noted that, as used in this specification and the appended claims, the singular forms “a”, “an”, and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific embodiments and techniques. However, it should be understood that many variations and modifications may be made while remaining within the spirit and scope of the invention.

### Further Embodiments

In an embodiment the invention includes a cryopreservative composition including a chaotropic agent and a kosmotropic agent. The chaotropic agent can be present in an amount sufficient to inhibit the formation of ice crystals intracellularly. The kosmotropic agent can be present in an amount sufficient to preserve the stability of cellular proteins. The chaotropic agent can be selected from the group consisting of urea and guanidinium chloride. The chaotropic agent can include urea. The kosmotropic agent can be selected from the group consisting of trimethylamine N-oxide, proline, ectoine, α,α-trehalose, glycine betaine, and 3-dimethylsulfoniopropionate. The kosmotropic agent can include trimethylamine-N-oxide. The ratio of the moles of the kosmotropic agent to the total moles of both kosmotropic agent and chaotropic agent can be equal to between about 0.1 to about 0.35. The ratio of the moles of the kosmotropic agent to the total moles of both kosmotropic agent and chaotropic agent can be equal to about 0.2. The total concentration of the kosmotropic agent in combination with the chaotropic agent can be equal to or less than about 1.5 moles per kilogram of solvent. The total concentration of the kosmotropic agent in combination with the chaotropic agent can be equal to between about 0.3 moles per kilogram of solvent and about 0.5 moles per kilogram of solvent.

In an embodiment, the invention includes a cryopreservative composition including urea and trimethylamine-N-oxide. The urea can be present in an amount sufficient to inhibit the formation of ice crystals intracellularly. The trimethylamine-N-oxide can be present in an amount sufficient to preserve the stability of cellular proteins. The molar ratio of trimethylamine-N-oxide can be equal to between about 0.1 to about 0.35. The molar ratio of trimethylamine-N-oxide can be equal to about 0.2. In some embodiments, the total concentration of the urea in combination with the trimethylamine-N-oxide can be equal to or less than about 1.5 moles per kilogram of solvent. The total concentration of the urea in combination with the trimethylamine-N-oxide can be equal to between about 0.3 moles per kilogram of solvent and about 0.5 moles per kilogram of solvent.

In an embodiment, the invention includes a method of cryopreserving cells. The method can include contacting cells with a cryopreservative composition, the cryopreservative composition can include a chaotropic agent and a kosmotropic agent. The chaotropic agent can be selected from the group consisting of urea and guanidinium chloride. The chaotropic agent can include urea. The kosmotropic agent can be selected from the group consisting of trimethylamine N-oxide, proline, ectoine, α,α-trehalose, glycine betaine, and 3-dimethylsulfoniopropionate. The kosmotropic agent can be trimethylamine-N-oxide.

In an embodiment, the invention includes a method of transplanting cells into a subject. The method can include administering a composition to the subject, the composition including an effective amount of a chaotropic agent, an effective amount of a kosmotropic agent, and cells. In an embodiment, the cells can include autologous cells. The chaotropic agent can be selected from the group consisting of urea and guanidinium chloride. The chaotropic agent can include urea. The kosmotropic agent can be selected from the group consisting of trimethylamine N-oxide, proline, ectoine, α,α-trehalose, glycine betaine, and 3-dimethylsulfoniopropionate. The kosmotropic agent can include trimethylamine-N-oxide.

1. A cryopreservative composition comprising:
a chaotropic agent; and
a kosmotropic agent.

2. The cryopreservative composition of claim 1, the chaotropic agent present in an amount sufficient to inhibit the formation of ice crystals intracellularly.

3. The cryopreservative composition of claim 1, the kosmotropic agent present in an amount sufficient to preserve the stability of cellular proteins.

4. The cryopreservative composition of claim 1, the chaotropic agent comprising urea.

5. The cryopreservative composition of claim 4, the urea present in an amount sufficient to inhibit the formation of ice crystals intracellularly.

6. The cryopreservative composition of claim 1, the kosmotropic agent selected from the group consisting of trimethylamine N-oxide, proline, ectoine, α,α-trehalose, glycine betaine, and 3-dimethylsulfoniopropionate.

7. The cryopreservative composition of claim 1, the kosmotropic agent comprising trimethylamine-N-oxide.

8. The cryopreservative composition of claim 7, the trimethylamine-N-oxide present in an amount sufficient to preserve the stability of cellular proteins.

9. The cryopreservative composition of claim 1, the ratio of the moles of the kosmotropic agent to the total moles of both kosmotropic agent and chaotropic agent equal to between about 0.1 to about 0.35.

10. The cryopreservative composition of claim 1, the ratio of the moles of the kosmotropic agent to the total moles of both kosmotropic agent and chaotropic agent equal to about 0.2.

11. The cryopreservative composition of claim 1, the total concentration of the kosmotropic agent in combination with the chaotropic agent equal to or less than about 1.5 moles per kilogram of solvent.

12. The cryopreservative composition of claim 1, the total concentration of the kosmotropic agent in combination with the chaotropic agent equal to between about 0.1 moles per kilogram of solvent and about 0.5 moles per kilogram of solvent.

13. A method of cryopreserving cells comprising:
contacting cells with a cryopreservative composition, the cryopreservative composition comprising a chaotropic agent and a kosmotropic agent.

14. The method of claim 13, the chaotropic agent comprising urea.

15. The method of claim 13, the kosmotropic agent selected from the group consisting of trimethylamine N-oxide, proline, ectoine, α,α-trehalose, glycine betaine, and 3-dimethylsulfoniopropionate.

16. The method of claim 13, the kosmotropic agent comprising trimethylamine-N-oxide.

17. A method of transplanting cells into a subject, the method comprising:
administering a composition to the subject, the composition comprising an effective amount of a chaotropic agent, an effective amount of a kosmotropic agent, and cells.

18. The method of claim 17, the cells comprising autologous cells.

19. The method of claim 17, the chaotropic agent comprising urea.

20. The method of claim 17, the kosmotropic agent selected from the group consisting of trimethylamine N-oxide, proline, ectoine, α,α-trehalose, glycine betaine, and 3-dimethylsulfoniopropionate.

21. The method of claim 17, the kosmotropic agent comprising trimethylamine-N-oxide.
